(19) United States
(12) Patent Application Publication
(10) Pub. No.: US 2001/0044534 A1
(43) Pub. Date: Nov. 22, 2001

(54) Pyrimidine derivative salt

(76) Inventor: Tomoya Kitazume, Tokyo (JP)

Correspondence Address: CROWELL & MORING LLP, INTELLECTUAL PROPERTY GROUP, P.O. BOX 14300, WASHINGTON, DC, 20044-4300, US

(21) Appl. No.: 09/799,097

(22) Filed: Mar. 6, 2001

(30) Foreign Application Priority Data
Mar. 6, 2000 (JP) 2000-061040

(51) Int. Cl. C07D 239/70

(52) U.S. Cl. 540/593; 544/282

(57) ABSTRACT

The invention relates to a compound represented by the general formula (1),

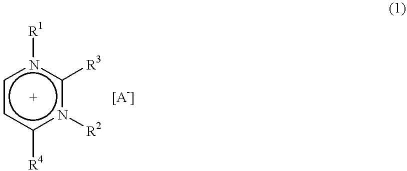

where R¹ is an alkyl group or trialkylsilyl group; R² is an alkyl group, or a combination of R² and R³ or R⁴ forms an alkylene group; each of R³ and R⁴ is a hydrogen or a portion of the alkylene group; and A⁻ is an anion. This compound (an ionic liquid) is useful as a catalyst and/or a reaction solvent in organic syntheses.

### BACKGROUND OF THE INVENTION

The present invention relates to ionic liquids, which are useful for organic syntheses and the like. Ionic liquids can serve as both of catalyst and solvent. Thus, much attention has recently been attracted in organic syntheses to ionic liquids. Ionic liquid can be defined as being a salt formed of an organic cationic species and an anionic species. Furthermore, it can be defined as being in the form of liquid at a temperature of about 100° C. or lower and as having a vapor pressure that is substantially zero at about room temperature (e.g., 25° C.). Thus, it can be defined as being a substance that is in the form of liquid until a high temperature (e.g., about 300° C.). Ionic liquid can be used as a solvent for organic and inorganic compounds, as a catalyst for alkylation, polymerization, oligomerization and the like, and as an extraction solvent for metals and organic matters.

Inorganic Chemistry, Vol. 35, No., 1996 discloses an ionic liquid, 1-ethyl-3-methylimidazolium trifluoromethanesulfonate (hereinafter referred to as [bmim][OTf] where [bmim] represents 1-ethyl-3-methylimidazolium, and [OTf] represents trifluoromethanesulfonate), and its analogous substances.

Green Chemistry 1, 23 (1999) discloses dienophiles (e.g., diethyl maleate, acrylonitrile, and ethyl acrylate) and a Diels-Alder reaction between a dienophile (e.g., cyclopentadiene, diethyl acetylenecarboxylate, and diethyl acrylate) and isoprene, using an ionic liquid (e.g., [bmim][OTf], [bmim][PF₆], [bmim][BF₄], and [bmim][lactate]) as catalyst and solvent.

### SUMMARY OF THE INVENTION

It is an object of the present invention to provide a novel compound having a catalytic activity.

According to the present invention, there is provided a compound represented by the general formula (1),

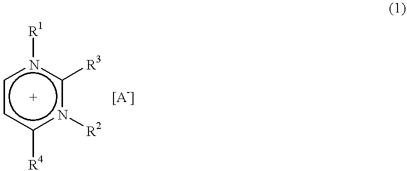

where R¹ is an alkyl group or trialkylsilyl group; R² is an alkyl group, or a combination of R² and R³ or R⁴ forms an alkylene group; each of R³ and R⁴ is a hydrogen or a portion of the alkylene group; and A⁻ is an anion.

### DESCRIPTION OF THE PREFERRED EMBODIMENTS

The compound represented by the general formula (1) can be defined as being an ionic liquid. In fact, it is a salt formed of (a) a heterocyclic ring cation containing a pyrimidine ring and (b) an anion species. The alkyl group for R¹ and R² may have a carbon atom number of about 1-22, preferably about 1-5, and is optionally branched. Nonlimitative examples of such alkyl group are methyl group, ethyl group, n-propyl group, isopropyl group, n-butyl group, n-pentyl group, and n-hexyl group. The trialkylsilyl group for R¹ contains three alkyl groups which are independent with each other. Each of these alkyl groups may have a carbon atom number of about 1-22, preferably about 1-5, and is optionally branched. Nonlimitative examples of such alkyl group can be the same as above. Examples of the trialkylsilyl group are trimethylsilyl group, triethylsilyl group, and dimethylethylsilyl group.

In the general formula (1), R² may be bonded with R³ or R⁴ to form an alkylene group —(CH₂)ₙ— where n is an integer that can be about 3-22, particularly preferably 3 or 5. Furthermore, a compound represented by the general formula (1), in which a combination of R² and R³ forms an alkylene group, is preferable to another compound in which a combination of R² and R⁴ forms an alkylene group. That is, a preferable compound is represented by the general formula (2),

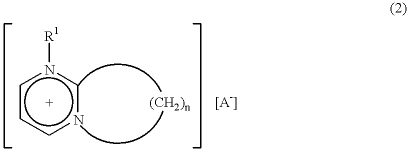

where R¹ is an alkyl group or trialkylsilyl group; n is an integer that may be of about 3-22, particularly preferably 3 or 5; and A⁻ is an anion. To this R¹, the above description for an alkyl group and trialkylsilyl group of the general formula (1) is applicable. In fact, an alkyl group concerning R¹ of the general formula (2) has a carbon atom number preferably of 1-10, more preferably of 1-5.

In the present specification, such cation may be abbreviated to bicyN⁺ (1,5-diazabicyclo[4,3,0]nonenium cation when n=3) or bicyU⁺ (1,8-diazabicyclo[5,4,0]-7-undecenium cation when n=5). The alkyl group may have a carbon atom number of about 1-22, preferably about 1-5, and is optionally branched. Nonlimitative examples of such alkyl group are methyl group, ethyl group, n-propyl group, isopropyl group, n-butyl group, n-pentyl group, n-hexyl group, and benzyl group. The trialkylsilyl group for R¹ contains three alkyl groups which are independent with each other. Each of these alkyl groups may have a carbon atom number of about 1-22, preferably about 1-5, and is optionally branched. Nonlimitative examples of such alkyl group can be the same as above. Examples of the trialkylsilyl group are trimethylsilyl group, triethylsilyl group, and dimethylethylsilyl group. The polymethylene group may have a carbon atom number of about 3-22, particularly preferably 3 or 5.

As stated above, the compound represented by the general formula (1) or (2) contains a heterocyclic ring cation and an anionic species (A⁻). This anionic species can be selected from a carboxylate anion represented by the general formula (3), an alkanesulfonate anion represented by the general formula (4), and a bis(alkanesulfonyl)amide anion represented by the general formula (5),

R′COO⁻ (3)

where R′ is an alkyl group or fluoroalkyl group,

R′SO₃⁻ (4)

where R′ is defined as above,

(R′SO₂)₂N⁻ (5)

where R′ is defined as above, or a combination of two of R′ is an alkylene group or fluoroalkylene group.

In the carboxylate anion represented by the general formula (3), the carbon atom number of R′ may be of about 1-10. Its examples include acetate anion, propionate anion, trifluoroacetate anion, pentafluoropropanoate anion, heptafluorobutanoate anion, and nonafluoropentanoate anion.

In the alkanesulfonate anion represented by the general formula (4), the carbon atom number of R′ may be of about 1-10. Its examples include methanesulfonate anion, ethanesulfonate anion, trifluoromethanesulfonate anion (hereinafter may be referred to as “TfO⁻”), pentafluoroethanesulfonate anion, 2,2,2-trifluoroethanesulfonate anion, heptafluoropropanesulfonate anion, 2,2,3,3,3-pentafluoropropanesulfonate anion, and nonafluorobutanesulfonate anion.

In the bis(alkanesulfonyl)amide represented by the general formula (5), the carbon atom number of R′ may be of about 1-10. Its examples include bis(trifluoromethanesulfonyl)amide anion (hereinafter may be referred to as “Tf₂N⁻”), bis(pentafluoroethanesulfonyl)amide anion, bis(heptafluoropropanesulfonyl)amide anion, bis(nonafluorobutanesulfonyl)amide anion, bis(undecafluoropentanesulfonyl)amide anion, (trifluoromethanesulfonyl)(pentafluoroethanesulfonyl)amide anion, (trifluoromethanesulfonyl)(heptafluoropropanesulfonyl)amide anion, (trifluoromethanesulfonyl)(nonafluorobutanesulfonyl) amide anion, bis(2,2,2-trifluoroethanesulfonyl)amide anion, (trifluoromethanesulfonyl)(2,2,2-trifluoroethanesulfonyl) amide anion, and an anion represented by the following formula:

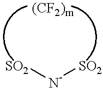

where m is an integer of 2-10.

Further examples of the anionic species include halogen ions and complex ions, for example, hexafluorophosphate, tetrachloroaluminate, and hexafluoroborate.

Of the above-mentioned exemplary anionic species, fluorocarboxylate anion, fluoroalkanesulfonate anion and bis(fluoroalkanesulfonyl)amide anion are preferable. Furthermore, perfluoroalkanesulfonate anion and bis(perfluoroalkanesulfonyl)amide anion are more preferable.

Preferable examples of the ionic liquid according to the invention include [5-methylbicyN⁺][TfO⁻], [5-ethylbicyN⁺][TfO⁻], [5-propylbicyN⁺][TfO⁻], [5-isopropylbicyN⁺][TfO⁻], [5-butylbicyN⁺][TfO⁻], [5-sec-butylbicyN⁺][TfO⁻], [5-tert-butylbicyN⁺][TfO⁻], [5-pentylbicyN⁺][TfO⁻], [5-methylbicyN⁺][Tf₂N⁻], [5-ethylbicyN⁺][Tf₂N⁻], [5-propylbicyN⁺][Tf₂N⁻], [5-isopropylbicyN⁺][Tf₂N⁻], [5-butylbicyN⁺][Tf₂N⁻], [5-sec-butylbicyN⁺][Tf₂N⁻], [5-tert -butylbicyN⁺][Tf₂N⁻], [5-pentylbicyN⁺][Tf₂N⁻], [8-methylbicyU⁺][TfO⁻], [8-ethylbicyU⁺][TfO⁻], [8-propylbicyU⁺][TfO⁻], [8-isopropylbicyU⁺][TfO⁻], [8-butylbicyU⁺][TfO⁻], [8-sec-butylbicyU⁺][TfO⁻], [8-tert-butylbicyU⁺][TfO⁻], [8-pentylbicyU⁺][TfO⁻], [8-methylbicyU⁺][Tf₂N⁻], [8-ethylbicyU⁺][Tf₂N⁻], [8-propylbicyU⁺][Tf₂N⁻], [8-isopropylbicyU⁺][Tf₂N⁻], [8-butylbicyU⁺][Tf₂N⁻], [8-sec-butylbicyU⁺][Tf₂N⁻], [8-tert-butylbicyU⁺][Tf₂N⁻], and [8-pentylbicyU⁺][Tf₂N⁻].

Particularly preferable examples of the ionic liquid according to the invention include those represented by the following formulas.

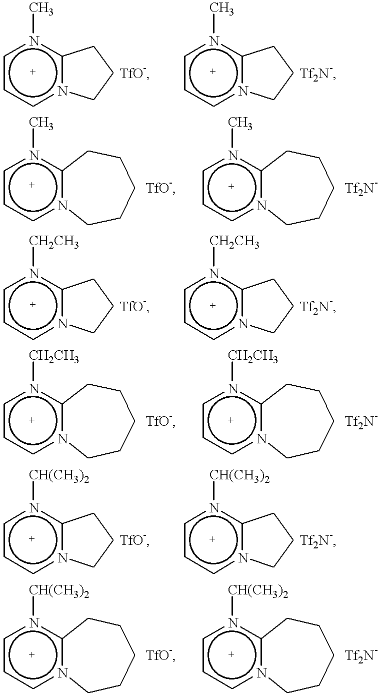

The process for producing the ionic liquid is not particularly limited. It is exemplarily described as follows. At first, a quaternary ammonium salt can be obtained by reacting a pyrimidine derivative, which is represented by the general formula (6), with an alkylation agent,

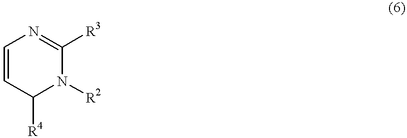

where R² is an alkyl group, or a combination of R² and R³ or R⁴ forms an alkylene group; each of R³ and R⁴ is a hydrogen or a portion of the alkylene group.

Typical examples of the alkylation agent are methyl chloride, ethyl chloride, methyl bromide, ethyl bromide, dimethyl sulfate, diethyl sulfate, and benzyl chloride. Furthermore, the alkylation agent can be selected from other conventional alkylation agents. The quaternary ammonium salt obtained by the alkylation may contain an anionic species such as a halogen ion (e.g., chlorine ion) or sulfate ion. Therefore, it is possible to obtain, for example, a trifluoromethanesulfonate by adding trifluoromethanesulfonic acid to the resulting quaternary ammonium salt to achieve ion-exchange of the anionic species. For example, an anionic species exchange from a halogen ion to bis(perfluoroalkanesulfonyl)amide ion can be achieved by reacting the quaternary ammonium salt with bis(trifluoromethanesulfonyl)amide lithium in water. Another anionic species exchange from a halogen ion to perfluoroalkanesulfonate ion can be achieved by reacting the quaternary ammonium salt with a potassium salt of perfluoroalkanesulfonic acid. A still another anionic species exchange from a halogen ion to a carboxylic acid ion or perfluorocarboxylic acid ion can be achieved by reacting the quaternary ammonium salt with a salt of a metal (e.g., sodium, potassium or silver). In this case, the ionic liquid can be obtained by removing an insoluble metal halide as a by-product. Furthermore, the ionic liquid can be obtained by only an alkylation using a particular alkylation agent, that is, an alkyl ester (e.g., ethyl trifluoromethanesulionate) of an acid (e.g., trifluoromethanesulfonic acid) corresponding to an anionic species of the ionic liquid. With this, the resulting ionic liquid can contain an anionic species of trifluorosulfonate or the like.

The novel compound according to the invention is useful as an ionic liquid. For example, it can be used as a catalyst and/or a solvent in syntheses of heterocyclic compounds, as shown in the after-mentioned Applied Examples.

The following nonlimitative examples are illustrative of the present invention.

### EXAMPLE 1

### Production of 8-ethyl-1,8-diazabicyclo[5,4,0]-7-undecenium=trifluoromethanesulfonato

At first, 35.6 g (0.2 mol) of ethyltrifluoromethanesulfonato (CF₃SO₃Et) were added at room temperature to 30.4 g (0.2 mol) of 1,8-diazabicyclo[5,4,0]-7-undecene in a reaction vessel, followed by stirring for 2 hr. Then, low-boiling-point substances were distilled out under reduced pressure, while the reaction vessel was put in an oil bath of 70° C. for 2 hr, thereby obtaining 8-ethyl-1,8- diazabicyclo[5,4,0]-7-undecenium=trifluoromethanesulfonato (yield: 98%). The analytical data of this target product were as follows.

Melting point (mp). 36-37° C. ¹HNMR (CDCl₃): δ1.28 (3H, t, J=7.32 Hz), 1.75-1.84 (8H, m) 2.15 (2H, m) 2.87 (2H, m) 3.55-3.59 (2H, m) 3.60 (2H, q, J=7.82 Hz) 3.68 (2H, m). ¹³CNMR (CDCl₃): δ13.554, 19.959, 22.841, 25.937, 28.069, 28.475, 46.210, 48.855, 48.968, 55.080, 120.584 (q, J=320.4 Hz), 165.966. ¹⁹FNMR (CDCl₃): δ83.37 (standard: C₆F₆). Elemental analysis: calculated values (C₁₂H₂₁F₃N₂SO₃: C (43.63), H (6.41), N (8.48)); measured values (C (43.43), H (6.41), N (8.50)).

### EXAMPLE 2

### Production of 8-methyl-1,8-diazabicyclo[5,4,0]-7-udecenium=trifluoromethanesulfonato

At first, 16.4 g (0.1 mol) of methyltrifluoromethanesulfonato (CF₃SO₃Me) were added at room temperature to 15.2 g (0.1 mol) of 1,8-diazabicyclo[5,4,0]-7-undecene in a reaction vessel, followed by stirring for 2 hr. Then, low-boiling-point substances were distilled out under reduced pressure, while the reaction vessel was put in an oil bath of 70° C. for 2 hr, thereby obtaining 8-methyl-1,8-diazabicyclo[5,4,0]-7-undecenium=trifluoromethanesulfonato (yield: 95%). The analytical data of this target product were as follows.

¹HNMR (CDCl₃): δ1.29 (3H, t, J=7.14 Hz), 2.13-2.81 (4H, m), 3.07 (2H, t, J=7.96 Hz), 3.48 (6H, m), 3.79 (2H, t, J=7.69 Hz). ¹³CNMR (CDCl₃): δ19.856, 22.182, 26.165, 28.649, 28.718, 41.203, 48.707, 48.847, 55.248, 120.459 (q, J=320.4 Hz), 166.516 ¹⁹FNMR (CDCl₃): δ83.37 (standard: C₆F₆).

### EXAMPLE 3

### Production of 5-ethyl-1,5-diazabicyclo[4,3,0]nonenium-trifluoromethanesulfonato

At first, 3.56 g (20 mmol) of ethyltrifluoromethanesulfonato (CF₃SO₃Et) were added at room temperature to 2.48 g (20 mmol) of 1,5-diazabicyclo[4,3,0]-5-nonene in a reaction vessel, followed by stirring for 2 hr. Then, low-boiling-point substances were distilled out under reduced pressure, while the reaction vessel was put in an oil bath of 70° C. for 2 hr, thereby obtaining 5-ethyl-1,5-diazabicyclo[4, 3,0]nonenium=trifluoromethanesulfonato (yield: 98%). The analytical data of this target product were as follows.

5 mp: 5-6° C. ¹HNMR (CDCl₃): δ1.29 (3H, t, J=7.14 Hz), 2.13-2.81 (4H, m), 3.07 (2H, t, J=7.96 Hz), 3.48 (6H, m), 3.79, t, J=7.69 Hz). ¹³CNMR (CDCl₃): δ12.450, 17.837, 18.680, 29.932, 41.961, 43.502, 47.944, 53.965, 120.456 (q, J=320.4 Hz), 165.686 ¹⁹FNMR (CDCl₃): δ83.23 (standard: C₆F₆).

### APPLIED EXAMPLE 1

### Synthesis of 4H, 2H-2-(phenyl)-3,1-benzoxazine

At first, 246 mg (2 mmol) of 2-aminobenzyl alcohol and 212 mg (2 mmol) of benzaldehyde were added to 1 g of 8-ethyl-1,8-diazabicyclo[5,4,0]-7-undecenium=trifluoromethanesulfonato, followed by stirring for 30 min at room temperature and then extraction with 20 ml of diethyl ether three times. Then, at least 99% of an ionic liquid of the lower layer was collected. The diethyl ether layer was dried with magnesium sulfate (MgSO₄), followed by distilling diethyl ether out, thereby isolating the target product (yield: 95%).

¹HNMR (CDCl₃): δ4.95 (1H, d, J=14.56 Hz), 5.35 (1H, d, J=14.56 Hz), 5.95 (1H, s), 6.70-7.60 (Ar—H).

¹³CNMR (CDCl₃): δ67.641, 85.126, 116.879, 119.596, 121.951, 124.831, 126.871, 127.247, 128.469, 128.890, 128.913, 138.913, 141.428, 159.5. Elemental analysis: calculated values (C₁₄H₁₃NO: C, (79.60), H, (6.20), N, (6.63)); measured values (C, (79.35), H, (6.45), N, (6.70)).

### APPLIED EXAMPLE 2

### Synthesis of 4H, 2H-2-(phenyl)-3,1-benzodiazine

At first, 244 mg (2 mmol) of 2-aminobenzylamine and 212 mg (2 mmol) of benzaldehyde were added to 1 g of 8-ethyl-1,8-diazabicyclo[5,4,0]-7-undecenium=trifluoromethanesulfonato, followed by stirring for 30 min at room temperature and then extraction with 20 ml of diethyl ether three times. Then, at least 99% of an ionic liquid of the lower layer was collected. The diethyl ether layer was dried with magnesium sulfate (MgSO₄), followed by distilling diethyl ether out, thereby isolating the target product (yield: 92%).

¹HNMR (CDCl₃): δ3.99 (1H, d, J=16.75 Hz), 4.27 (1H, d, J=16.75 Hz), 5.24 (1H, s), 6.57-7.53 (Ar—H).

The entire disclosure of Japanese Patent Application No. 2000-061040 filed on Mar. 6, 2000, including specification, claims and summary, is incorporated herein by reference in its entirety.

What is claimed is:

1. A compound represented by the general formula (1), 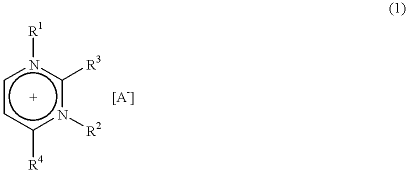
where R¹ is an alkyl group or trialkylsilyl group; R² is an alkyl group, or a combination of R² and R³ or R⁴ forms an alkylene group; each of R³ and R⁴ is a hydrogen or a portion of the alkylene group; and A⁻ is an anion.

2. A compound represented by the general formula (2), 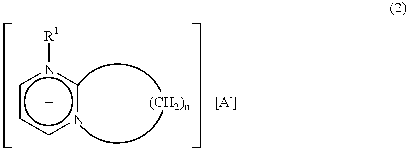
where R¹ is an alkyl group or trialkylsilyl group, n is an integer, and A⁻ is an anion.

3. An ionic liquid according to claim 1, wherein said anion is selected from the group consisting of a carboxylate anion represented by the general formula (3), an alkanesulfonate anion represented by the general formula (4), and a bis(alkanesulfonyl)amide anion represented by the general formula (5),
R′COO⁻ (3)
where R′ is an alkyl group or fluoroalkyl group,
R′SO₃⁻ (4)
where R′ is defined as above,
(R′SO₂)₂N⁻ (5)
where R′ is defined as above, or a combination of two of R′ is an alkylene group or fluoroalkylene group.

4. A compound according to claim 2, wherein said integer is from about 3 to about 22.

5. A compound according to claim 4, wherein said integer is 3 or 5.

6. A compound according to claim 1, wherein said R¹ has a carbon atom number of 1-10.

7. A compound according to claim 1, wherein said anion comprises a fluorocarboxylate anion, a fluoroalkanesulfonate anion, or a bis(fluoroalkanesulfonyl)amide anion.

8. A compound according to claim 1, wherein said anion comprises a perfluoroalkanesulfonate anion or a bis(perfluoroalkanesulfonyl)amide anion.

9. A compound according to claim 1, which is represented by one of the following formulas, 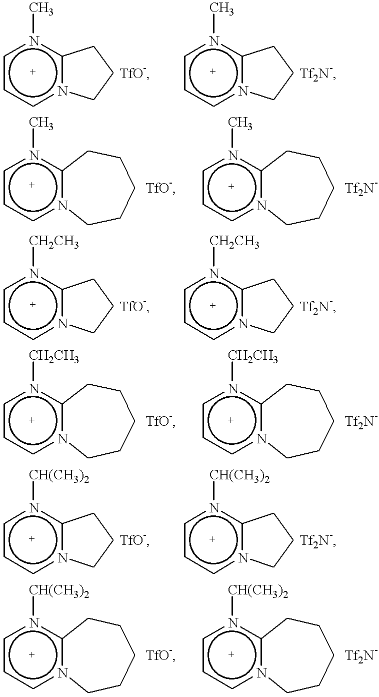
where TfO⁻ represents a trifluoromethanesulfonate anion, and Tf₂N⁻ represents a bis(trifluoromethanesulfonyl)amide anion.

10. A process for producing a compound represented by the general formula (1), 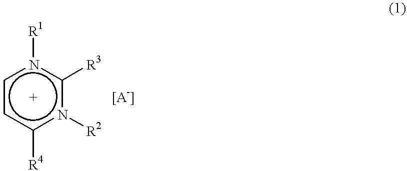
where R¹ is an alkyl group or trialkylsilyl group; R² is an alkyl group, or a combination of R² and R³ or R⁴ forms an alkylene group; each of R³ and R⁴ is a hydrogen or a portion of the alkylene group; and A⁻ is an anion,
said process comprising reacting a pyrimidine derivative, which is represented by the general formula (6), with an alkylation agent, 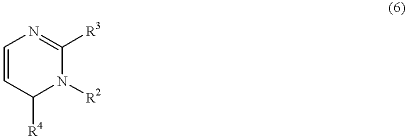
where R², R³ and R⁴ are defined as above.

11. A process according to claim 10, further comprising subjecting a product of said reacting to an anion exchange.

12. A process according to claim 15, wherein said alkylation agent comprises an anionic species that is identical with said anion of said compound.
